(19) United States
(12) Patent Application Publication
(10) Pub. No.: US 2024/0001210 A1
(43) Pub. Date: Jan. 4, 2024

(54) Interactive Exercise Paddling System

(71) Applicants: Bradley John Byron Galvin, Burnaby (CA); Alan John Bojsza, Campbell River (CA); Svetozar Ristovski, Vancouver (CA)

(72) Inventors: Bradley John Byron Galvin, Burnaby (CA); Alan John Bojsza, Campbell River (CA); Svetozar Ristovski, Vancouver (CA)

(21) Appl. No.: 18/254,165

(22) PCT Filed: Jul. 16, 2022
(86) PCT No.: PCT/CA2022/051109

§ 371 Date: May 23, 2023

Related U.S. Application Data
(60) Provisional application No. 63/222,973, filed on Jul. 17, 2021.

(51) Int. Cl. A63B 69/00; A63B 71/06

(52) U.S. Cl. CPC A63B 69/0093; A63B 71/0622; A63B 2220/13; A63B 2220/806; A63B 2220/808; A63B 2071/0625; A63B 2225/50; A63B 2071/0638

(57) ABSTRACT

Examples of a interactive paddling system are described. The interactive paddling system comprises a paddling assembly, a display screen connected to the paddling assembly, a plurality of sensors configured to detect a position and/or orientation of the paddleboard, the pole and/or the user and generate output signals, a user interface operable to receive input information from the user and provide output and a processing system communicably coupled to the paddling assembly, the display screen, the user interface and the plurality of sensors to receive, from the user interface, the user input data and output signals from the plurality of sensors and to send the output signals to the actuator of the stability adjusting system to adjust a position and orientation of the paddleboard and to display and track a performance parameters of the user in real time during a particular activity content.

### TECHNICAL FIELD

The present disclosure generally relates to an interactive exercise system and more particularly to an interactive exercise paddling system.

### BACKGROUND

Paddle boarding is an activity that is enjoyed by many people. One difficulty with the activity of paddle boarding is that it is often limited to periods of nice weather such as summer or warm and sunny days. This is particularly problematic in colder and norther climates.

Attempts have been previously provided to train a user in the use an operation of a paddle board on land. However, such attempts have been overly large and complicated and have not accurately replicated both the movement of the paddle through the water when paddle boarding. Nor have such attempts replicated the balance requirements of operating a paddle board. Examples of such attempts may be found in U.S. Pat. No. 8,708,867 to Lumsden et al. and US Patent Application Publication Nos. 2014/0221169 to Bourne et al., 2017/0165552 to Martin and 2011/0275489 to Apau.

Unless otherwise indicated herein, the materials described in this section are not prior art to the claims in this application and are not admitted to be prior art by inclusion in this section.

### SUMMARY

In one aspect, an interactive paddling system is provided. The interactive paddling system comprises a paddling assembly that includes a paddleboard supporting a user. The paddleboard has a body with a top surface and a bottom surface, an endless belt positioned along a periphery of the body, a plurality of engagement elements in communication with the endless belt, a pole configured to engage the engagement elements, a stability adjustment system and an actuator. The interactive paddling system further comprises a display screen operably connected to the paddling assembly, a plurality of sensors configured to detect a position and/or orientation of the paddleboard, the pole and/or the user and generate output signals, a user interface operable to provide input and output information from and to the user and a processing system communicably coupled to the paddling assembly, the display screen, the user interface and the plurality of sensors to receive, from the user interface, the user input data and output signals from the plurality of sensors and to send the output signals to the actuator of the stability adjusting system to adjust a position and orientation of the paddleboard and to display and track a performance parameters of the user in real time during a particular activity content.

In another aspects a first processing system is communicably coupled to the first paddling assembly, the first display screen, the first user interface and the plurality of sensors, such that the first processing system is operable to display, on the display screen, information identifying a plurality of activity content accessible by the first processing system through a communication network, receive, from the first user interface, a first user selected activity, output to the display screen the selected activity content comprising a digital video content and an audio content, track the first user performance parameters received from the plurality of sensors at a particular point in the selected activity, display on the display screen at least one of the first user performance parameters, and display on the display screen at least one of a plurality of second user performance parameters received via the digital communication network from a second processing system at a second location associated with a second interactive paddling system, wherein at least one of first user performance parameters at the particular point in the selected activity and at least one of the second user performance parameters at the same point in the selected activity are presented for comparison on the display screen at the first location.

In addition to the aspects and embodiments described above, further aspects and embodiments will become apparent by reference to the drawings and study of the following detailed description.

### BRIEF DESCRIPTION OF THE DRAWINGS

Throughout the drawings, reference numbers may be re-used to indicate correspondence between referenced elements. The drawings are provided to illustrate example embodiments described herein and are not intended to limit the scope of the disclosure. Sizes and relative positions of elements in the drawings are not necessarily drawn to scale. For example, the shapes of various elements and angles are not drawn to scale, and some of these elements are arbitrarily enlarged and positioned to improve drawing legibility.

FIG. 1 illustrates an example of an interactive exercise paddling system showing a user standing on a paddling device with a pole and a display.

FIG. 2 is a perspective view of a bottom of a paddleboard in an example of an interactive paddling system of a present invention.

FIG. 3a is a cross-sectional view of the paddleboard of FIG. 2 showing an adjustable support in stable position.

FIG. 3b is a cross-sectional view of the paddleboard of FIG. 2 showing an adjustable support in unstable (rocking) position.

FIG. 4 is an exploded view of another embodiment of an interactive exercise padding system with an endless belt and a belt stabilizing system.

FIG. 5 is a perspective view of the paddleboard of FIG. 4 showing the endless belt and belt stabilizing system in assembled configuration.

FIGS. 6a and 6b illustrate a perspective partial view of the paddleboard of FIGS. 4-5 showing a belt stabilizing system with a track engrossing the belt and a wheel system mounted in the track in slidable fashion.

FIGS. 6c-6d illustrate side cross-sectional view of a belt stabilizing system.

FIGS. 7a-7b illustrate an example of a wheel system of a belt stabilizing system.

FIG. 8 illustrates another example of an interactive exercise paddling system.

FIG. 9 illustrates an example of a system for interactively connecting multiple interactive exercise paddling systems.

FIG. 10 is an example of a user interface according to an embodiment of the present disclosure.

FIG. 11 is an example of a graphic user interface according to an embodiment of the present disclosure.

FIG. 12 is a schematic view of an example of visual image display of an exercise activity using an interactive exercise paddling system.

### DETAILED DESCRIPTION OF SPECIFIC EMBODIMENTS

The present invention discloses an interactive exercise paddling system that a user can use to take interactive live or archived paddling classes or paddleboard yoga classes at the convenience of their home.

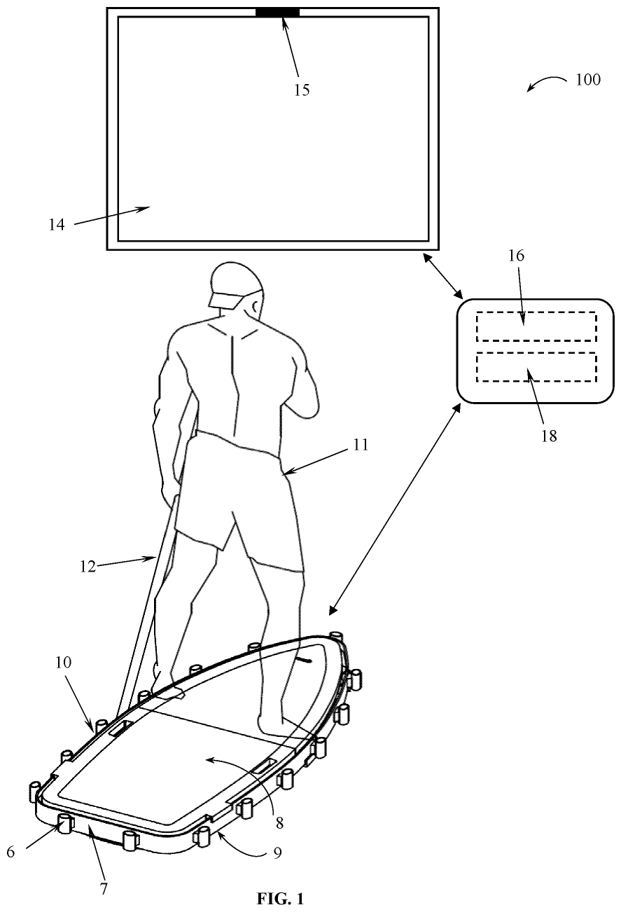

FIG. 1 illustrates an example of an interactive paddling system 100 showing a paddleboard 10 upon which a user (paddler) 11 can stand, a pole/paddle 12 and a display 14. The pole 12 can comprise an elongated shaft portion and a handle designed for the paddler 11 to grip. Various embodiments of the paddleboard 10 are described in the co-pending U.S. Pat. No. 10,881,935 which is included herein in entirety by reference. The paddleboard 10 has a body with a top surface 8, a bottom surface 9 and an endless band or belt 7 extending around a peripheral edge of the paddleboard 10 along a path substantially perpendicular to the top surface 8 and slidably supported by the paddleboard 10 for rotational movement thereabout. A plurality of engaging elements 6 are coupled to the endless belt 7 at a predetermined distance one from another along the endless belt 7. Each of the plurality of engaging elements 6 can comprise an opening and are configured such that the pole 12 can engage with it moving the endless belt 7 simulating a paddling activity. For example, the shaft of the pole 12 is selectively engageable with one of the engaging elements 6 to rotate the belt 7 relative to the body of the paddleboard 10 when the paddler 11 pulls back on the pole 12. The belt 7 may be formed from any suitable flexible material, such as, by way of non-limiting example, rubber, fabrics or the like. For example, the belt 7 may be formed from fabric reinforced conveyor belt such as, by way of non-limiting example, PVC conveyor belt or the like. The belt 7 is configured to engage with the pole 12 to move the peripheral member of the paddleboard 10 about its body. The belt 7 is moveable in a first direction, for example counter-clockwise, and a second direction clockwise. Moving the belt 7 in the first direction mimics paddling on the right side of the paddleboard 10, while moving the belt 7 in a second direction mimics paddling on the left side of the paddleboard 10.

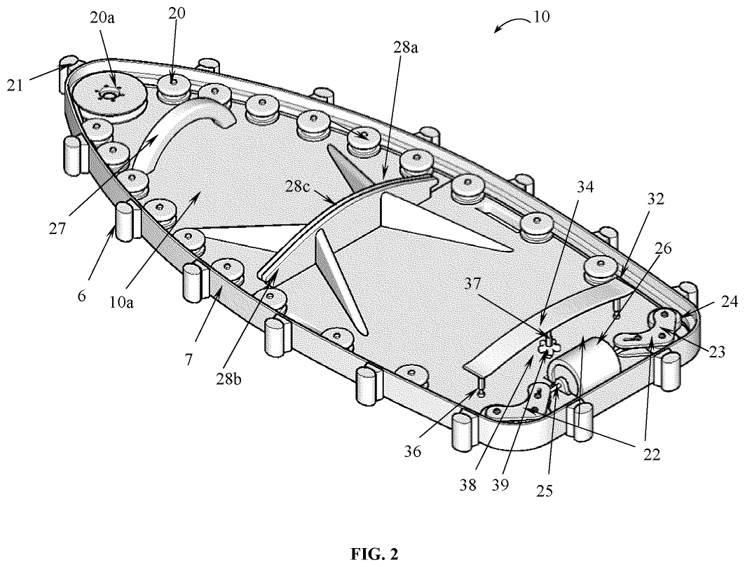

As illustrated in FIG. 2, the underside of the body 10a of the paddleboard 10 includes a plurality of rollers 20 therearound along the periphery of the body 10a. The rollers 20 support the belt 7 to maintain it along the shape of the paddleboard's body as well as keep the belt 7 at a desired height relative to the body. A larger lead roller 20a may be positioned proximate to the front edge 21 of the paddleboard 10 and corner rollers may be positioned to each side of the rear edge of the paddleboard 10. It will be appreciated that such larger rollers may be stronger to bear a greater tension in the belt 7 as well as to form more gradual bends in the path of the belt 7. The rollers 20 may be rotatably connected to and supported by the body 10a by any known means, such as, by way of non-limiting example, bushings, bearings or the like. The paddleboard 10 further comprises a tension adjustment mechanism 22 that allows selective adjustment of the tension of the belt 7. This enables the paddler 11 seen in FIG. 1 to adjust the force required to move the belt 7 relative to the body of the paddleboard 10 and thus enables the paddler 11 to adjust the degree of difficulty of the workout when exercising. The tension adjustment mechanism 22 comprises an actuator that may be of any type known to a person skilled in the art. For example, the tension adjustment mechanism 22 as illustrated may comprise rotating arms 23 supported with tensioning rollers 24, a connector 25 and an actuator 26. The connector 25 may comprise a slot in the arm 23 through which a threaded rod and a thumb wheel are passed. The threaded rod is secured to the body such that tightening of the thumb wheel fixes the location of the threaded rod in the slot so as to fix the orientation of the rotating arm and thereby the distance by which the tensioning roller 24 is extended from the paddleboard's body 10a. Any other tension adjustment mechanism 22 can be used to adjust the tension of the belt 7 without departing from the scope of the invention. The tension adjustment actuator 26 can be selected from an electric actuator, a pneumatic actuator, a hydraulic actuator or any mechanical actuator. In some implementations, the one or more of the rollers and/or the lead roller 20a may include a brake to adjust the force required by a user to rotate the belt 7. The brake may include a brake shoe having a braking surface positioned to engage upon and frictionally limit rotation of the lead roller 20a. An actuation button can be used to actuate the brake.

The paddleboard 10 may also include a plurality of guides/tracks 112 (not shown) extending from a peripheral edge of the body adapted to support the belt 7. For example, the paddleboard 10 may include one or more guides on each side of the front portion of the paddleboard 10 as are necessary to support the belt 7. The front guides can be configured to hold the belt 7 up so as to prevent movement of the user's pole 12 from pushing the belt 7 off the rollers 20 at the start of their stroke. One or more guides can also be located in the rear portion of the body 10a to hold the belt down so as to prevent lifting of the belt 7 by the user's pole during the follow through of their stroke.

The paddleboard 10 further comprises a stability adjustment system to adjust a stability of the body 10a of the paddleboard 10 and thus enables the paddler 11 to adjust the degree of difficulty of the workout when exercising using the system 100. The stability adjustment system can comprise an arcuate member 27 with an arcuate surface along at least portion of the member 27 and located at the front portion of the paddleboard 10, and a front wall 28a and a rear wall 28b that also have an arcuate surface 28c. The arcuate surfaces of the member 27 and the walls 28a, 28b permit the body 10a to swing/rotate on a support surface such as a floor so as to permit the body 10a to rock during operation by the user 11. The stability adjustment system also includes an adjustable support 32 in the rear portion of the body 10a. The adjustable support 32 may be configured to have a similar concave profile as the arcuate member 27 and the walls 28a, 28b or may optionally have its curvature adjusted to reduce the stability of the body 10a to swing. It will be appreciated that although the adjustable support 32 is located in the rear portion, it may also be located in any other location or more than one of the supports may be adjustable as described herein.

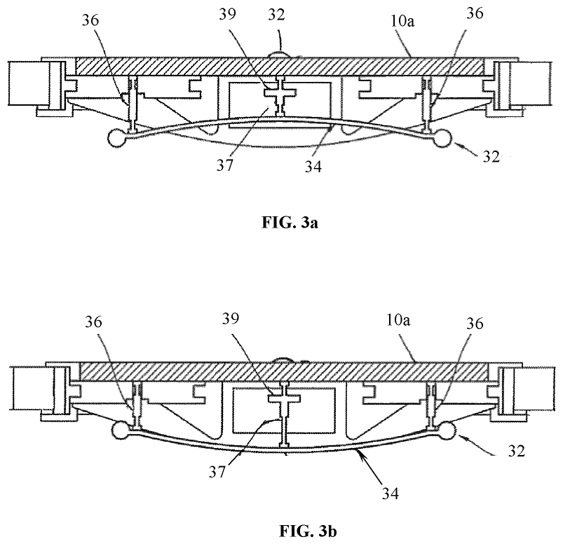

The adjustable support 32 is formed of a flexible elongate member 34 extending transversely to the axis of the paddleboard 10. The flexible member 34 is supported proximate to each end with a fixed support 36. An adjuster 38 is located at a middle portion of the flexible member 34. The adjuster 38 comprises a threaded rod 37 extending from the flexible member 34 which is received in a wheel 39 supported by the body 10a. As the wheel 39 rotates, the threaded rod 37 is telescoping adjusted therein so as to adjust the length of the rod 37 and thereby the curvature of the flexible member 34. As illustrated in FIG. 3a, the adjustable support 32 is shortened so as to cause the flexible member 34 to have a concave profile in which the apparatus will be more stable. As illustrated in FIG. 3b, the adjustable support 32 is lengthened so as to cause the flexible member 34 to have a convex shape thereby making the apparatus less stable. In such a manner, a user may select the degree of stability they wish for the paddleboard 10 to have. An actuator can be operably coupled to the wheel 39 to actuate the wheel 39 and thus adjust the length of the rod 37 and thus the curvature of the flexible member 34. The actuator can be selected from an electric actuator, a pneumatic actuator, a hydraulic actuator or a mechanical actuator. In one embodiment, the actuator can be a linear actuator.

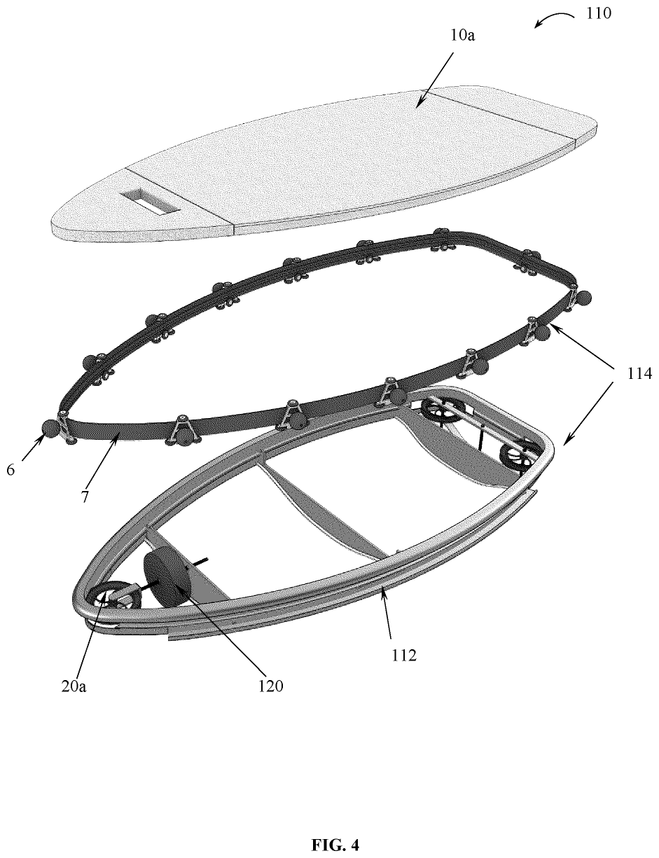

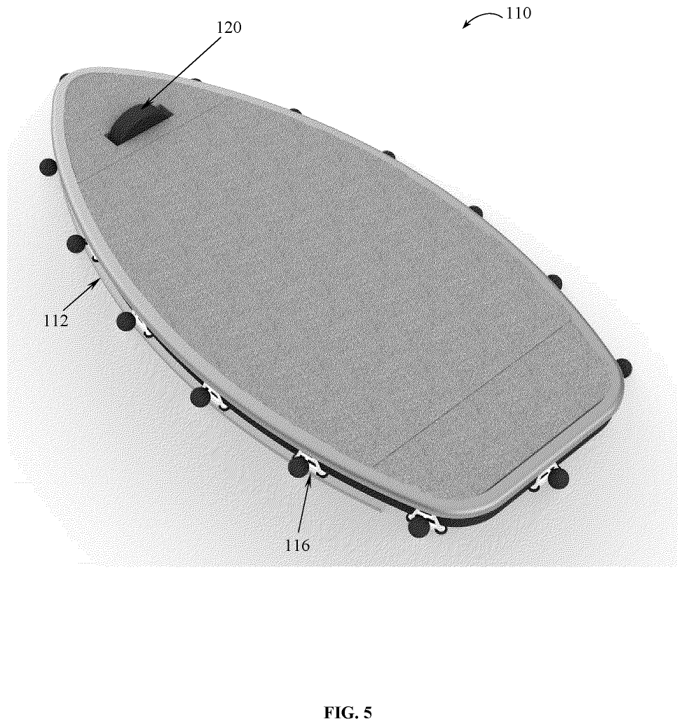

FIGS. 4 and 5 show another example of an interactive exercise paddling system 110 similar to system 100 additionally comprising a belt stabilizing system 114. The belt stabilizing system 114 comprises a track 112 and a wheel system 116. The conveyor belt 7 requires rigidity through the paddle stroke while being flexible navigating the corners of the paddle board body 10a. For example, in some implementations, the belt 7 flexibility can cause derailment of the belt 7 from the rollers 20 due to the lateral engagement of the pole 12 upon the engagement elements 6. The belt stabilizing system 114 comprises the wheel system 116 engaging the track 112 that is engrossing the conveyor belt 7 so that wheels 113 are fit in positions and roll along the track 112 to prevent friction while providing stability to the belt 7 that is receiving lateral pressure from the paddle/pole 12. This will prevent the rollers 20 (now omitted) on the platform causing distortion when paddled and the belt derailing. As illustrated in FIG. 5, the track 112 is attached to the body 10a of the paddleboard 110, the belt 7 positioned and engrossed in the track 112 and the wheel system 116 mounted on the track 112 such that wheels 113 can slide along track guides. The system 110 can further comprise a belt tensioning wheel/roller 120 that can protrude through the top surface 8 of the body 10a and which is configured to extend the front roller 20a to tension the belt 7.

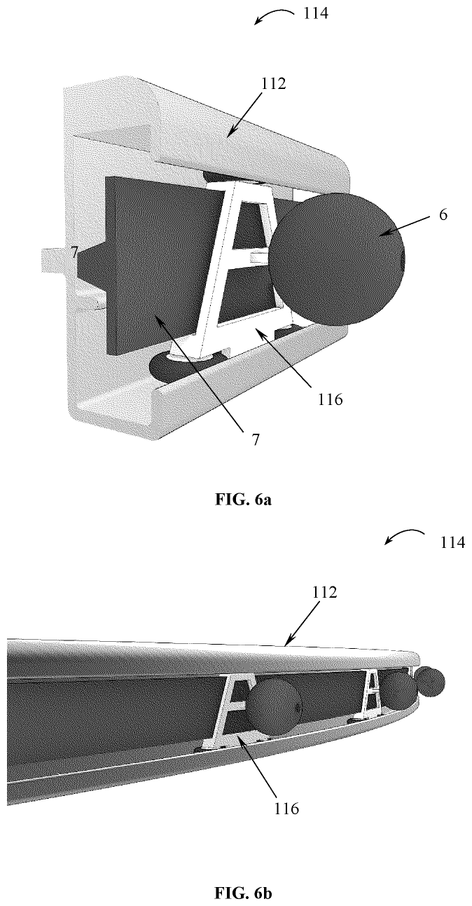

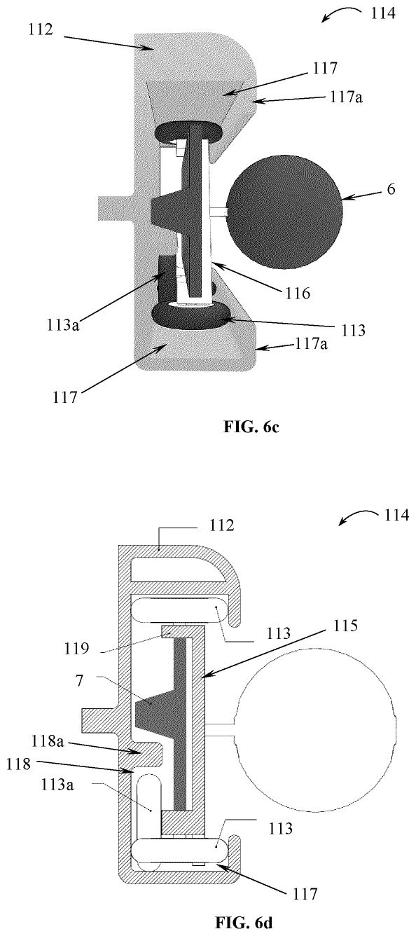

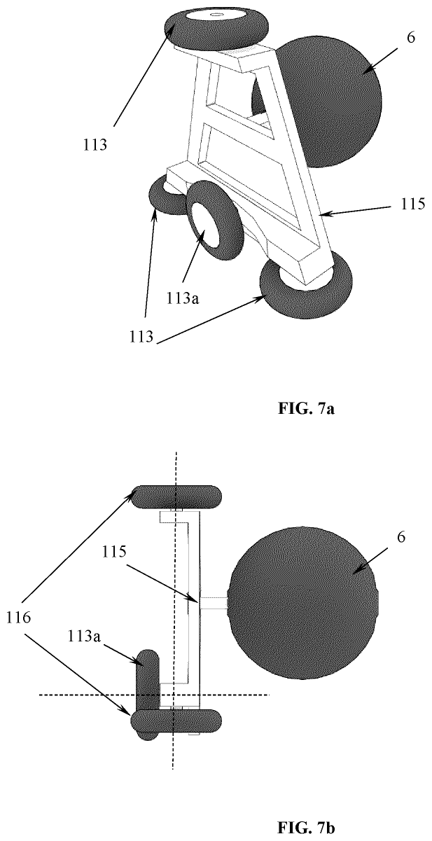

FIGS. 6a-6d illustrates in detail an example of the belt stabilizing system 114 in assembled configuration showing an example of the wheel system 116 and the track 112. The wheel system 116 comprises a carrying body 115 carrying a set of wheels 113 and the paddle engaging element 6. In the illustrated assembly there are three wheels 113 mounted in a triangle configuration, each of the wheels 113 having an axis of rotation that is parallel one to another, and an additional wheel 113a with an axis of rotation perpendicular to the axes of rotation of the wheels 113 (see FIGS. 7a and 7b). The track 112 is configured to provide a first track guide 117 with a ledge 117 to secure and provide guide for the wheels 113 and a second track guide 118 with a ledge 118a to secure and guide the wheel 113a. The carrying body 115 further can comprise a bracket 119 to rivet a longitudinal edge portion of the belt 7 and further control belt derailment and its stability during paddling activity. By positioning and securing the wheel system 116 within the track 112 which is also engrossing the belt 7, it is secured control and stability of the conveyor belt 7 during paddling while also preventing friction. Person skilled in the art would understand that fewer or more wheels 113, 113a can be mounted to the carrying body 115 in various configuration and adapted to slide along the track guides without limiting the scope of the invention.

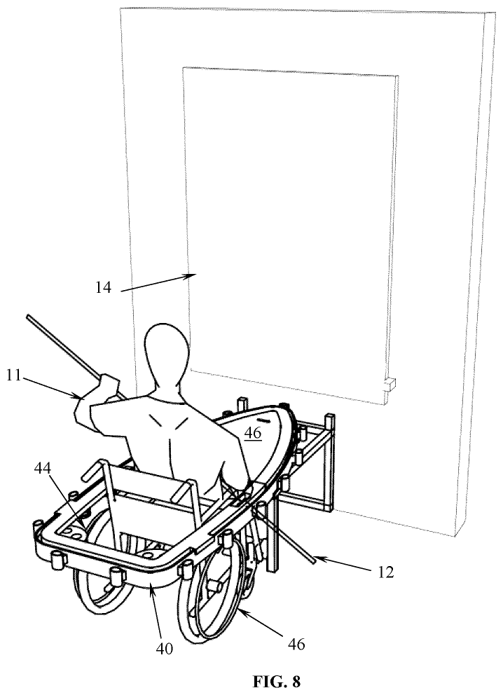

FIG. 8 shows another example of an interactive exercise paddling system which is similar to the system 100 of FIG. 1 or system 110 of FIG. 5 with the following exceptions. The system comprises a paddleboard 40 with a body 42 that has a central aperture 44 positioned between the front and rear of the paddleboard and extending between the sides of the paddleboard 40. The aperture 44 is shaped to receive a paddler 11 in a wheelchair 46 therethrough. The body 42 is shape to receive and selectively couple to the wheelchair 46.

The display 14 can be mounted away of the paddleboard 10, such as for example can be mounted on a wall. In one implementation the display 14 can be a smart mirror. The display 14 can be connected to the paddleboard 10 with wires or wirelessly, via for example a Bluetooth®. In one implementation, the display 14 can be mounted to the body 10a of the paddleboard 10.

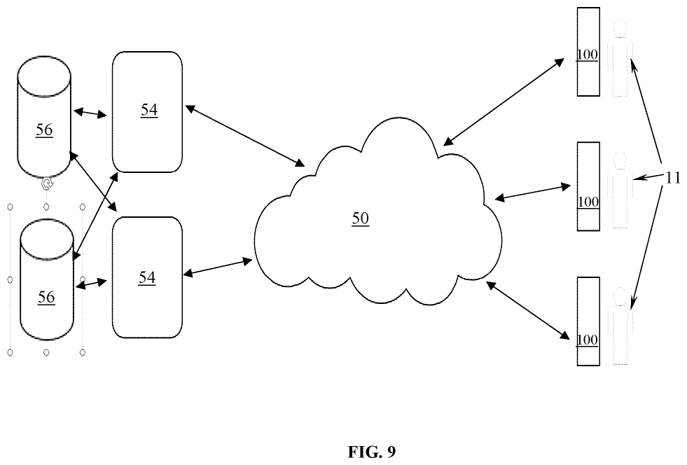

Digital components can be connected to or integrated with the interactive paddleboard 10. Some of the digital components can be located remotely and connected to the paddleboard 10 and/or the display 14 wirelessly. The digital components may include memory unit or digital storage, processing unit, communications hardware, software, antennas, cameras, microphones, keyboards, touchscreens, joysticks, headsets, VR/AR devices and/or audio speakers. In some implementations, the digital components may be integrated with the paddleboard 10. In some embodiments, the system 100, 110 may include application programs such as smart devices associated apps and/or web-based application programs that provide access to exercise content, such as live or archive paddling or yoga classes, geolocation maps, simulations and holograms, as well as user's account performance information. The exercise contents, geolocation maps, simulations and holograms, as well as user's account performance information, can be stored locally on a personal computing device or on remote servers 54 connected to the paddling system 100, 110 through a wirelessly communication network 50 (FIG. 9). The computing devices, e.g., servers 54 can be further in communication with one or more databases 56 that store for example a predetermined content, such as for example, pre-recorded class content that can be broadcasted to the system 100, 110 on demand or as a live class. Any other information (e.g., game content, simulations, holograms, etc.) can be stored in the database and can be accessible by the servers 54 and the systems 100, 110 through the network 50.

Plurality of sensors can be mounted or in communication with the paddling system 100, 110. For example, a number of sensors such as directional port sensor, directional starboard sensor, an accelerometer, an inertial measurements unit etc. can be in communication with the paddleboard 10 to detect a position, orientation of the paddleboard and/or speed of the endless belt movement. In addition, the paddling system can comprise a camera 15 that can be mounted on the display 14 to capture a position of the paddleboard 10 as well as the position of the paddler 11 standing upon the paddleboard. Any other sensors such as pressure sensors, tension sensors, speakers, microphones can be connected to the system 100, 110.

In addition, a biometric sensor can also be provided to detect physiological parameters of the user 11 during the activity, such as paddleboard yoga class or paddling class. For example, the biometric sensor can measure the hearth rate, breath rate, wetness of the skin, respiration, hydration, or any other physical characteristic of the user 11 during activity. The sensors can be wearables worn by the user 11 (incorporated in a smart wearable device such as smart watch or smart glasses), or be connected to the paddleboard 10 or the pole 12. The signals detected by the sensors can be received as an input by a processing system 16 and stored in a memory unit 18. The processing system 16 and/or the memory unit 18 can be positioned in a personal computing device connected to the paddling assembly and/or the display by wires or wirelessly. In some implementations, the processing system 16 and/or the memory unit 18 can be in remote servers 54 connected to the paddling system 100, 110 through a wirelessly communication network 50 and can communicate to the paddleboard 10 and the display 14 through one or more wireless connections. The processing system 16 can be programed to calculate and store a range of performance information, activity level, such as for example, distance, speed, tension and power, wave height, water current, direction, wind speed, cadence, heart rate, respiration, hydration, calorie burn, and/or any custom performance scores can be calculated as current/instantaneous values, maximum, minimum, average, or total over time, or using any other statistical analysis.

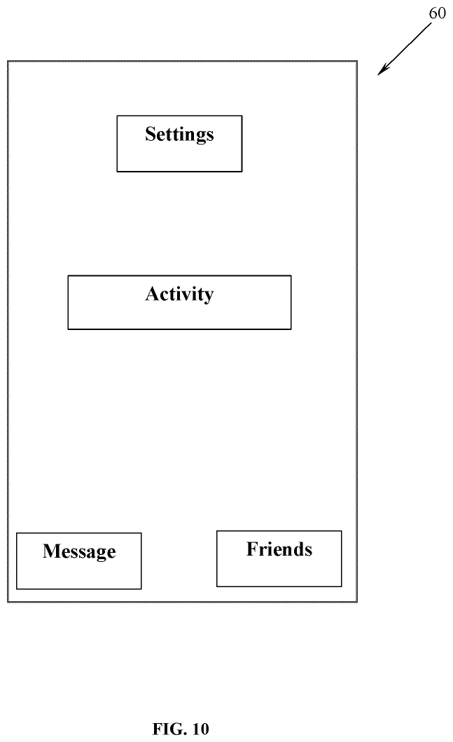

A user interface 60 (FIG. 10) is configured to receive input from the user 11 as well as provide output to the user. The user interface 60 is programmed with executable instructions for obtaining input data from the user, transmitting data to the processing system 16 and in some embodiments to the memory unit 18 and to provide output data generated by the processing system 16 and/or any connected computing device. The processing system 16 can receive input data from the user interface 60 as well as detected signals from the plurality of sensors and can generate output signals to for example the actuator of the adjustable support 32 to adjust a position and orientation of the paddleboard 10 and to the display screen 14 to display and track performance parameters in real time during a particular activity content. In some implementation, the processing system 16 can provide a recommendation to the user as an output using the user interface 60 and/or the display 14. The system 100, 110 also comprises a power supply system that is operably coupled and configured to power the paddling assembly, the display screen, the plurality of sensors, the user interface and the processing system. The power supply can comprise two or more separate power supplies.

The interactive paddling system 100, 110 can be wirelessly connected to one or more servers 54 with memory storing units via one or more wireless connections. FIG. 9 illustrates two remote servers 54 that can have an access to one or more databases 56 and may be storing at least one of an archived activity content and users' performance and physiological parameters. One or more interactive paddling systems 100, 110 can be operably coupled to the one or more servers 54 and/or databases 56 over a communication network 50. The one or more servers 54 can store a first user's performance parameters in each particular point of the activity content and the second user's performance parameters in each particular point of the same activity content and can generate an output signal to the first display screen of the first interactive paddling system to display the first user's performance parameters and the second user's performance parameters and an output signal to the second display screen of the second interactive paddling system to display the second user's performance parameters and the first user's performance parameters. The at least one of the first user performance parameters at the particular point in the selected activity and the at least one of the second user performance parameters at the same point in the selected activity are presented for comparison on the first display screen at the first location and vice versa. In some implementations, the processing system can generate a ranking list based on performance parameters of the first and second users and display such list on the respective display of the first and second paddling system. The ranking list can dynamically change based of the user's performance parameters in a particular point of the activity. The first paddling system 100, 110 can comprise a first wireless connection to operably couple to the one or more servers 54 via the communication network 50 and the second paddling system 100, 110 can comprise a second wireless connection to operably couple to the one or more servers 54 via the communication network 50.

The activity content can comprise a digital video content and an audio content. The activity content can be a live or archived paddling/yoga class content or a game content that can be produced to the respective display screen. In some implementations, the display screen 14 can display the activity content and a video imagery of an exercise instructor. In some embodiments, the display screen 14 can also display biometric information of the first user and in some implementations the biometric information of the second user too. In some implementations, the video content can comprise a simulated dynamic environment or geolocation or terrain with a number of simulated environments and obstacles.

The user interface can comprise a graphical user interface with user selectable content that can be displayed on the display screen during operation of the paddling system 100, 110. For example, the display screen 14 can be driven by the user input device such as a touchscreen, mouse, or other device. In one implementation, the user interface can comprise an application running on a smart device such as a smartphone, smart watch or a tablet. FIG. 10 shows an example of a graphical user interface 60. The graphical user interface 60 can be installed on any smart device, including smartphones, smart watches, personal computers on any suitable operating system such as IOS, Windows, Android, etc. The user interface 60 can include a settings tab for users' customizations, such that users can input their pre-set preferences. The interface 60 can further comprise an activity tab to select preferred activity from a list of archived or live activities. The user interface can include a number of different options, such as to send an invitation to a number of different users to a particular activity or to follow activities of different users, as well as to send/share a video/audio or text messages with one or more users. The user interface 60 can provide a wide range of control and informational tabs/windows that can be accessed and removed by a click, touch, or gesture. In some embodiments, the windows may provide information about the user's own past performance parameters and/or the performance of other users in the same class. The user interface 60 can be used to access user membership information and settings, login and logout of the system, access live and archived activity content, friend network etc. The user interface 60 can provide direct links to content such as a link to join a particular activity. The user can navigate among the different screens in the user interface by selecting such links using the input device such as by touching the touchscreen at the indicated location, or by swiping to bring on a new screen.

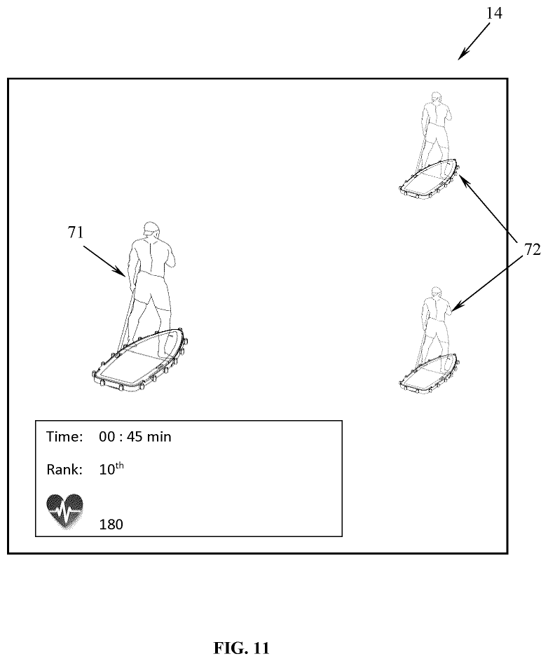

The user interface 60 can also be used to access and update profile or member information, manage account settings such as information sharing, and control device settings. For example, user interface 60 can be used to select information and arranging how the information is presented on the display screen 14. FIG. 11 shows one example of the display screen 14 showing the selected information and arranging as selected by the user. For example, the display screen can show an image of the first user 71 performing the selected activity, such as for example a live paddling class as well as an image of at least one additional paddler 72 participating in the same live class. The display screen can also present some of the user's performance parameters in comparison to the performance parameters of the other paddlers performing the class (e.g., showing the current rank list of the first user), user's biometric information, such as for example the heart rate, how long the user was paddled, the paddling speed, etc. The user interface can be preset such that any other information and arranging can be displayed on the screen 14 without departing from the scope of the invention.

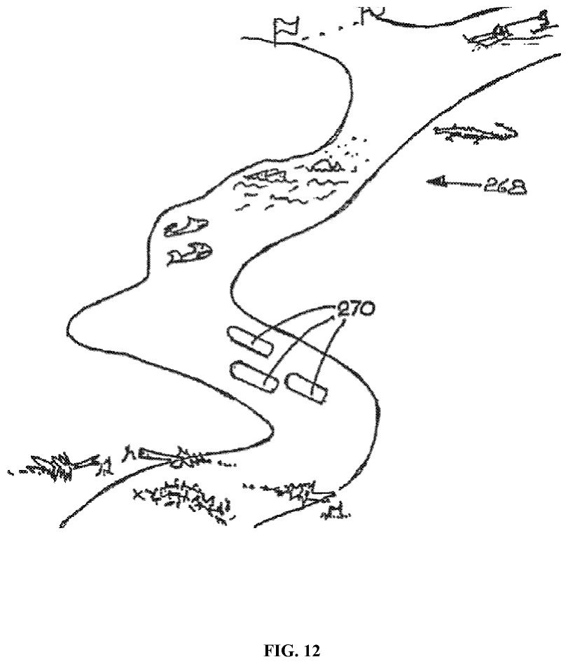

In some implementation, the users and/or any other organizer/instructor can create competitions by all users or just a selected subset of users, such as for example a group of friends. Competitions can be created within a class or session, or across multiple classes or sessions like multi-stage paddling races. In addition, other types of exploration activities or games can be created and simulated. For example, FIG. 12 shows a visual display of a generated simulated environment 268, such as a river with a number of obstacles. Each of the paddlers participating in the activity are shown as a virtual paddling avatar 270. The user can select the level of difficulty using the user interface or the level of difficulty and obstacles are dynamically changed by the processing system (based on the preprogramed game parameters). The processing system (which in this example can be a gaming console) can be configured to send signals to selectively actuate actuator of the tension system and the adjustable stability support based on the simulated environment and thereby adjust the degree of difficulty of the workout the degree of stability of paddleboard.

Various aspects and advantages of the embodiments have been described where appropriate. It is to be understood that not necessarily all such aspects or advantages may be achieved in accordance with any particular embodiment. Thus, for example, it should be recognized that the various embodiments may be carried out in a manner that achieves or optimizes one advantage or group of advantages as taught herein without necessarily achieving other aspects or advantages as may be taught or suggested herein.

1. An interactive paddling system comprising:
a paddling assembly including a paddleboard supporting a user having a body with a top surface and a bottom surface, an endless belt positioned along a periphery of the body, a plurality of engagement elements in communication with the endless belt, a pole configured to engage the engagement elements, a stability adjustment system and an actuator;
a display screen operably connected to the paddling assembly;
a plurality of sensors configured to detect a position and/or orientation of the paddleboard, the pole and/or the user and generate output signals;
a user interface operable to receive input from the user and provide output information, the user interface programmed with executable instructions for obtaining input data, transmitting data and generating output data;
a processing system with a memory unit, the processing system communicably coupled to the paddling assembly, the display screen, the user interface and the plurality of sensors, the processing system operable to receive, from the user interface, the user input data and from the plurality of sensors the output signals, and send output signals to the actuator to adjust a position and orientation of the paddleboard, the display screen to display and track a performance parameters in real time during a particular activity content and/or to the user interface; and
a power supply system operably coupled and configured to power the paddling assembly, the display screen, the plurality of sensors, the user interface and the processing system.

2. The interactive paddling system of claim 1, wherein the memory unit stores a library of archived exercise classes, geolocation maps, simulations and holograms.

3. The interactive paddling system of claim 1, further comprising one or more of an antenna, a camera, a speaker, a microphone, a VR device or an AR device.

4. The interactive paddling system of claim 1, wherein the paddling assembly further comprising a belt stabilizing system configured to stabilize the endless belt, the belt stabilizing system having a track mounted to the periphery of the body and having at least one track guide and a wheel system with a set of wheels mounted to the track and at least one track guide, the belt stabilizing system engrossing the endless belt.

5. The interactive paddling system of claim 4, wherein the paddling assembly further comprising a tension adjustment system with a tension actuator, the tension adjustment system coupled to the endless belt to adjust a tension of the endless belt and thus adjust a force required to move the endless belt relative to the paddleboard.

6.-8. (canceled)

9. The interactive paddling system of claim 1, wherein the display screen is mounted on the paddling assembly.

10. The interactive paddling system of claim 1, wherein the display screen is a smart display mounted away from the paddling assembly.

11. The interactive paddling system of claim 1 wherein the display screen is a touch screen.

12. The interactive paddling system of claim 1, wherein the user interface comprises a smart device running an application.

13. The interactive paddling system of claim 1, wherein the user interface comprises a graphical user interface with user selectable content for display during operation of the paddling system.

14. The interactive paddling system of claim 1, wherein the activity content comprising a digital video content and/or an audio content.

15. The interactive paddling system of claim 14, wherein the digital video content is a simulated dynamic environment.

16. (canceled)

17. The interactive paddling system of claim 1, wherein the activity content is selected from a live or an archived paddling class content or a live or an archived yoga class content.

18. (canceled)

19. The interactive paddling system of claim 15, wherein the activity content is produced to the display screen.

20. The interactive paddling system of claim 17, wherein the display screen displays the activity content and a video imagery of an exercise instructor.

21. The interactive paddling system of claim 1, wherein the plurality of sensors comprises one or more of directional port sensor, directional starboard sensor, an accelerometer, camera, pressure sensor, an inertial measurements unit or a biometric sensor.

22. The interactive paddling system of claim 21, wherein the display screen di splays biometric information of the user.

23. The interactive paddling system of claim 1, further comprising one or more servers with memory storing at least one of an archived activity content and user's performance parameters in each particular point of the activity content and a communication network, the interactive paddling system operably coupled to the one or more servers over the communication network.

24. (canceled)

25. The interactive paddling system comprising:
a first paddling assembly including a first paddleboard supporting a first user having a body with a top surface and a bottom surface, an endless belt positioned along a periphery of the body, a plurality of engagement elements in communication with the endless belt, a pole configured to engage the engagement elements, a first stability adjustment system and an actuator;
a first user display screen operably connected to the first paddling assembly to display activity content to the first user at a first location;
a first user interface comprising a first user input device operable to receive input information from the first user and provide output information;
a first plurality of sensors operable to detect activity by the first user and generate first user performance parameters; and
a first processing system communicably coupled to the first paddling assembly, the first display screen, the first user interface and the first plurality of sensors, the first processing system operable to display, on the display screen, information identifying a plurality of activity content accessible by the first processing system through a communication network, receive, from the first user interface, a first user selected activity, output to the display screen the selected activity content comprising a digital video content and an audio content, track the first user performance parameters received from the plurality of sensors at a particular point in the selected activity, display on the display screen at least one of the first user performance parameters, and display on the display screen at least one of a plurality of second user performance parameters received via the digital communication network from a second processing system at a second location associated with a second interactive paddling system, wherein at least one of first user performance parameters at the particular point in the selected activity and at least one of the second user performance parameters at the same point in the selected activity are presented for comparison on the display screen at the first location.

26. The interactive paddling system comprising:
a first paddling assembly including a first paddleboard supporting a first user having a body with a top surface and a bottom surface, an endless belt positioned along a periphery of the body, a plurality of engagement elements in communication with the endless belt, a pole configured to engage the engagement elements, a first stability adjustment system and a first actuator; a first user display screen operably connected to the first paddling assembly to display activity content to the first user at a first location; a first user interface comprising a first user input device operable to receive input information from the first user and provide output information; and a first plurality of sensors operable to detect activity by the first user and generate first user performance parameters;
a second paddling assembly including a second paddleboard supporting a second user having a body with a top surface and a bottom surface, an endless belt positioned along a periphery of the body, a plurality of engagement elements in communication with the endless belt, a pole configured to engage the engagement elements, a second stability adjustment system and a second actuator; a second user display screen operably connected to the second paddling assembly to display activity content to the second user at a second location; a second user interface comprising a second user input device operable to receive input information from the second user and provide output information; and a second plurality of sensors operable to detect activity by the second user and generate second user performance parameters;
one or more servers with memory storing at least one of an archived activity content, the first user's performance parameters in each particular point of the activity content and the second user's performance parameters in each particular point of the activity content; and
a communication network comprising a first wireless connection to operably couple the first paddling assembly to the one or more servers and a second wireless connection to operably couple the second paddling assembly to the one or more servers.
